(19) United States
(12) Patent Application Publication
(10) Pub. No.: US 2006/0073200 A1
(43) Pub. Date: Apr. 6, 2006

(54) Lercanidipine capsules

(75) Inventors: Amedeo Leonardi, Milan (IT); Fabio Berlati, Rho (IT); Lino Pontello, Milan (IT)

Correspondence Address: DARBY & DARBY P.C., P. O. BOX 5257, NEW YORK, NY, 10150-5257, US

(73) Assignee: Recordati Ireland Limited, Ringaskiddy (IE)

(21) Appl. No.: 11/244,315

(22) Filed: Oct. 4, 2005

Related U.S. Application Data
(60) Provisional application No. 60/615,919, filed on Oct. 5, 2004.
(60) Provisional application No. 60/656,792, filed on Feb. 25, 2005.

(51) Int. Cl. A61K 31/455; A61K 9/48

(52) U.S. Cl. 424/451; 514/355

(57) ABSTRACT

The invention provides a modified release lercanidipine pharmaceutical composition comprising at least one waxy substance and a therapeutically effective amount of lercanidipine, wherein oral administration of the modified release lercanidipine pharmaceutical composition to a patient results in a mean lercanidipine plasma concentration of greater than 0.5 ng/ml for the full time period of about 24 hours after administration of the composition to the patient.

### FIELD OF THE INVENTION

The present invention relates to solid modified release pharmaceutical compositions and solid oral dosage forms comprising lercanidipine and at least one waxy substance.

### BACKGROUND OF THE INVENTION

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with a long duration of action and high vascular selectivity. It has a high affinity for and competitively antagonizes the dihydropyridine subunit of the L-type calcium channel.

Lercanidipine is useful as an anti-hypertensive. Lercanidipine lowers blood pressure by blocking calcium channels of arterial smooth muscle, thus decreasing peripheral vascular resistance. Lercanidipine produces no negative cardiac inotropism and only occasionally, mild reflex tachycardia generally of short duration. Lercanidipine has been approved for the treatment of hypertension and has been marketed since 1996 in several European countries under the trademark Zanidip®.

The hydrochloride salt of lercanidipine is commercially available from Recordati S.p.A. (Milan, Italy). Methods of preparing lercanidipine hydrochloride, as well as methods of resolving lercanidipine into individual enantiomers are described in U.S. Pat. Nos. 4,705,797; 5,767,136; 4,968,832; 5,912,351; and 5,696,139, and U.S. Patent Publications No. 2003/0069285 and No. 2003/0083355, all of which are incorporated herein by reference.

Lercanidipine, alone or in combination with additional active agents, is effective in once and twice daily administration. Lercanidipine has been studied in dosages ranging from 2 to 80 mg. Lercanidipine is typically administered as an immediate release tablet at a dose of about 10 mg to about 20 mg once daily or twice daily. Lercanidipine is used for treating Stage I and Stage II hypertension and is also useful in alleviating angina pectoris. Lercanidipine is also beneficial in elderly patients with isolated systolic hypertension. The recommended starting oral dose of lercanidipine HCl is 10 mg once daily and is increased after at least 2 weeks, if necessary, to 20 mg daily. Upon oral administration of an immediate release form of lercanidipine, peak plasma level (Tₘₐₓ) occurs 1-3 hours following administration. Following administration of immediate release lercanidipine dosage forms, the plasma level of lercanidipine typically falls below 1 ng/ml by 24 h.

Lercanidipine and its salts are virtually insoluble in water, with an aqueous solubility of about 5 μg/ml. The solubility of lercanidipine is marginally greater in acidic media, however, even at pH 5 it is less than 20 μg/ml. Lercanidipine solubility at a pH greater than 5 is essentially less than 5 μg/ml. Thus, lercanidipine is essentially insoluble in gastrointestinal pH range of 1 to 8. Lercanidipine is also poorly permeable (Pₐₐₚ of 0.5×10⁻⁷ cm/s in a Caco-2 cell apparatus and low bioavailability) and is classified as a low permeable drug, as defined by the FDA. Additionally, lercanidipine displays extensive presystemic first pass elimination, as a result of its being a substrate for cytochrome P450 IIIA4 isoenzyme. The combination of poor water solubility, low permeability and considerable first pass metabolism results in low and highly variable bioavailability when lercanidipine is administered to a patient.

In order to improve the bioavailability of lercanidipine, food may be co-administered with each dosage. The administration of food along with lercanidipine has been shown to increase the absorption of lercanidipine significantly and therefore enhance its efficacy, a phenomenon known as “food effect.” Simultaneous intake of food (especially food having a high fat content) increases the amount of lercanidipine absorbed by three to four fold compared to administration without food. Lercanidipine administered in the absence of food is not entirely absorbed which results in low and variable bioavailability. The dependence of effective dosing and absorption of lercanidipine upon co-administration of food is undesirable due to fluctuations in effectiveness, inter-patient variability, and poor patient acceptance and compliance.

To facilitate the effective administration of lercanidipine alone or in combination with other active agents to patients, there is a need in the art for improved lercanidipine oral dosage forms. Lercanidipine oral dosage forms should have properties that overcome the difficulties caused by the low solubility of lercanidipine in aqueous media, allowing for simple formulation. Lercanidipine oral dosage forms should also achieve good lercanidipine absorption and bioavailability and provide at least a minimum effective lercanidipine plasma level over a period of at least 24 h.

Accordingly, the present inventors have discovered modified release pharmaceutical compositions and dosage forms comprising lercanidipine and at least one waxy substance that overcome the formulation and dosaging problems associated with prior art lercanidipine dosage forms. These new compositions are easily formulated and are adaptable to all forms of lercanidipine, e.g., free base, crystalline, amorphous, crystalline polymorphs, salts, solvates and oils. The compositions and dosage forms also provide greater bioavailability of lercanidipine, over an extended duration, and lower variability in dosaging compared to currently available lercanidipine compositions. Further, the present compositions are expected to eliminate or show a less pronounced food effect, based on data contained herein.

### SUMMARY OF THE INVENTION

The compositions of the present invention provide for modified release of lercanidipine, such that the mean plasma concentration of lercanidipine is greater than at least 0.5 ng/ml for the full time period of about 24 hours after administration of the composition to a patient.

In one embodiment, the present invention provides for a solid modified release lercanidipine pharmaceutical composition comprising at least one waxy substance and a therapeutically effective amount of lercanidipine, wherein oral administration of the modified release lercanidipine pharmaceutical composition to a patient results in a mean plasma concentration of lercanidipine of greater than about 0.5 ng/ml for the full time period of about 24 hours after administration of the composition, per 20 mg dose of lercanidipine. Preferred waxy substances are polyalcohol fatty acid esters, e.g., polyethylene or polypropylene glycol esters and glycerides, and combination thereof. More preferred waxy substances are polyglycolized glycerides.

In another aspect, the present invention provides a unit solid dosage form comprising a gelatin or hydroxypropylmethylcellulose capsule, at least one waxy substance and a therapeutically effective amount of lercanidipine dispersed in said waxy substance. Preferred waxy substances are those described above.

In other aspects, the present invention provides pharmaceutical compositions comprising at least one polyglycolized glyceride and a therapeutically effective amount of lercanidipine dispersed in said polyglycolized glyceride, wherein the polyglycolized glyceride has a melting point from about 40° C. to about 60° C. and a hydrophobic lipophilic balance (HLB) value from about 1 to about 14.

In a preferred embodiment, the invention provides a modified release pharmaceutical composition comprising at least one polyglycolized glyceride and a therapeutically effective amount of lercanidipine, wherein the polyglycolized glyceride is selected from the group consisting of Gelucire® 37/02, 37/06, 42/12, 44/14, 46/07, 48/09, 50/02, 50/13, 33/01, 39/01, 43/01, and 53/10, or a combination thereof.

The present invention also provides for a method of preparing a modified release lercanidipine solid unit dosage form, comprising the steps of: (i) heating a polyglycolized glyceride from about 40° C. to about 90° C., (ii) mixing the heated polyglycolized glyceride and lercanidipine to form a solution, and (iii) filling a capsule with the solution.

In other embodiments, the present invention provides a method of treating hypertension in a patient in need thereof comprising administering to said patient a therapeutically effective amount of lercanidipine in any of the aforementioned modified release formulations, to treat said hypertension.

### DESCRIPTION OF THE DRAWINGS

FIGS. 1A and 1B depicts the in vivo S-lercanidipine plasma concentrations in dogs resulting from administration of 40 mg lercanidipine in formulation Y1 (□) and Y2 (⋄) and reference formulation (∘).

FIG. 2 depicts in vitro dissolution profiles of lercanidipine modified release formulations Y1 (●) and Y2 (□).

FIG. 3 depicts a flow chart for preparation of lercanidipine modified release formulations.

### DETAILED DESCRIPTION OF THE INVENTION

As used herein, the following terms are defined as follows:

The term “lercanidipine” refers to the free base composition methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate, and pharmaceutically acceptable salts thereof. Pharmaceutically acceptable salts include, but are not limited to lercanidipine salts formed with inorganic or organic acids, such as (i) inorganic acids, such as hydrochloric acid, hydrobromic acid, phosphoric acid and sulfuric acid; (ii) sulfonic acids, such as methanesulfonic acid, benzenesulfonic acid, toluenesulfonic acid, and napthalene-1,5,-disulfonic acid; (iii) monocarboxylic acids, such as acetic acid, (+)-L-lactic acid, DL-lactic acid, DL-mandelic acid, gluconic acid, cinnamic acid, salicylic acid, and gentisic acid, (iv) dicarboxylic acids, such as oxalic acid, 2-oxo-glutaric acid, malonic acid, (−)-L-malic acid, mucic acid, (+)-L-tartaric acid, fumaric acid, maleic acid, and terephthalic acid, (v) tricarboxylic acids, such as citric acid, and (vi) aromatic sulfonimides such as saccharin. Preferred pharmaceutically acceptable salts of lercanidipine, include but are not limited to, the hydrochloride, besylate and napadisylate salts. Lercanidipine may be present in one or more crystalline or amorphous forms. Additionally, lercanidipine may be present as one or both of its enantiomeric forms.

As used herein, the term “amorphous” refers to a solid compound having no substantial crystal lattice structure. In one preferred embodiment, amorphous compounds are identified by DSC analysis. Typically, amorphous compounds have DSC plots with broad endothermic transitions, defined as glass transition, rather then sharp exothermic peaks typical of crystalline compounds. Additionally, amorphous compounds present XRD spectra having broad shoulders rather than well-defined peaks profile, which are characteristic of the crystalline solids.

The term “modified release” refers to release of the active ingredient, lercanidipine, from a composition of the present invention over a period of time sufficient to maintain therapeutically effective plasma levels over similarly extended time intervals and/or to modify other pharmacokinetic properties of the active ingredient. Preferably modified release provides for therapeutic plasma concentrations of lercanidipine for a period for about 20 to about 25 hours and a mean plasma concentration of lercanidipine of greater than 0.5 ng/ml, and preferably greater than 1 ng/ml, over the duration of the dosing interval.

The term “bioavailability” refers to the rate and extent to which the active ingredient or active moiety, e.g., lercanidipine, is absorbed from a drug product, e.g., capsule, and becomes systematically available.

As used herein, the term “pharmaceutically acceptable” refers to a biologically or pharmacologically compatible for in vivo use. Preferred pharmaceutically acceptable salts are those substances that are approved by a regulatory agency of the Federal or a state government or listed in the U.S. Pharmacopoeia or other generally recognized pharmacopoeia for use in animals, and more particularly in humans.

The term “therapeutically effective amount” refers to the amount of active agent sufficient to lower the blood pressure of a patient with hypertension. Therapeutically effective amounts of active agent preferably lower blood pressure, such that the values for systolic and diastolic blood pressure are below 140 and 90 mm Hg, respectively. A therapeutically effective amount of the active agent may or may not decrease the blood pressure in a person that does not have hypertension or may not decrease blood pressure in all persons with hypertension. Therapeutic effectiveness in treatment of other pathologies, such as heart failure or atherosclerosis is also specifically contemplated as per, e.g., U.S. Pat. Nos. 5,696,139 and 5,767,136, which are incorporated herein by reference. Preferably, a therapeutically effective amount of active agent leads to a reduction in blood pressure, e.g., within about 2 to 6 hours. Preferably, when a rapid reduction in blood pressure is desired, a therapeutically effective amount of active agent will reduce systolic blood pressure in the range from about 20-30 mm Hg and diastolic blood pressure in the range from about 10-20 mm Hg, within about 30 minutes to about 60 minutes following administration of the active agent.

The modified release lercanidipine pharmaceutical compositions of the present invention provide for modified release of lercanidipine over an extended period of time providing an increased mean plasma concentration of lercanidipine over the dosing duration, compared to commercially available lercanidipine compositions. In particular, when administered to a patient, the present compositions result in a mean plasma concentration of lercanidipine of greater than about 0.5 ng/ml for at least about 24 hours following administration. Without being bound by a particular theory, it is believed that the improved bioavailability of the present compositions, compared to commercially available tablets, is attributable to the presence of a non-aqueous waxy matrix component.

The term “waxy substance” refers to a plastic solid substance with a low melting point. “Waxy substance” may refer to one compound, one type of compound or a mixture of different compounds, as context requires. Waxy substances may be lipophilic or hydrophilic. Preferred waxy substances are polyalcohol fatty acyl esters, e.g., polyethylene glycol, polypropylene glycol esters and fatty acid glycerides, and combinations thereof. More preferred waxy substances are polyglycolized glycerides.

The term “solid” as used herein refers to a substance that is solid or semi-solid at room temperature. Hence, as used herein, a “solid” substance may become liquid at, e.g., body temperature.

Fatty acid glycerides suitable for use in modified release formulations include both medium chain and long chain fatty acid glycerides. In one aspect, the pharmaceutical compositions of the present invention may include one or more long chain (C₁₂ to C₂₂) fatty acid glycerides (including monoesters, diesters and/or triesters of glycerol). Examples of long chain fatty acid glycerides, suitable for use in the invention are glyceryl behenate(e.g., Compritol® 888 ATO) and glyceryl palmitostearate (e.g., Precirol® ATO 5). Compritol® 888 ATO and Precirol® ATO 5 are commercially available from Gattefosse Corporation, Paramus, N.J.).

Additional preferred fatty acid glycerides, suitable for use herein include one or more medium chain fatty acid glycerides such as one or more triglycerides of C₈ to C₁₁ fatty acids. An example of a medium chain fatty acid triglyceride, suitable for use in the invention is Miglyol® 812 (commercially available from Condea Chemie GmbH, Cranford, N.J.).

Polyethylene glycol esters and polypropylene esters suitable for use in modified release formulations include mono- and diesters of polyethylene glycols and polypropylene glycols. Suitable and preferred fatty acids for inclusion in polyethylene glycol esters and polypropylene glycol esters are C₁₂ to C₂₂ fatty acids, as set forth above. Suitable polyethylene glycol chains and polypropylene chains for use respectively in polyethylene glycol esters and polypropylene glycol esters are described in, e.g., the U.S. Pharmacopeia.

Preferred fatty acid glycerides for use in the modified release compositions of the invention, have a melting point of from about 40° C. to about 80° C. and a HLB value from about 1 to about 14, preferably from 10-14.

“Polyglycolized glycerides” denotes a mixture of mono-, di- and triglycerides and polyethylene glycol (PEG) mono- and diesters. Polyglycolized glycerides are particularly preferred waxy substances suitable for use in the present invention. Polyglycolized glycerides are commercially available under the name Gelucire® (Gattefosse Corporation, Paramus, N.J.).

Particular grades of Gelucire® which are useful in the present invention, include, but are not limited to Gelucire® 37/02, 37/06, 42/12, 44/14, 46/07, 48/09, 50/02, 50/13, 33/01, 39/01, 43/01 and 53/10, or combinations thereof. The first number in the nomenclature of a Gelucire® denotes its melting point while the second number characterizes its HLB value. For example, Gelucire® 50/13 has a melting point of about 55° C., and an HLB value of about 13. Particularly preferred grades of Gelucire®, are Gelucire® 50/13, and Gelucire® 44/14 or combinations thereof.

The modified release composition of the present invention comprises lercanidipine. The compositions may comprise any form of lercanidipine, e.g., crystalline, amorphous, crystalline polymorphs, salts, solvates and oils thereof.

In one embodiment, lercanidipine is provided as a salt of lercanidipine. A particularly preferred salt is lercanidipine hydrochloride.

Lercanidipine may be present in crystalline or amorphous forms, or a mixture of amorphous and crystalline forms, wherein the crystalline can be of the same polymorph or a combination of two or more crystalline polymorph forms. Crystalline forms of lercanidipine include, for example, those disclosed in U.S. Published Patent Applications No. 2003/0083355 and 2003/0069285, which are incorporated herein by reference. Preferred lercanidipine hydrochloride polymorphs are crystalline Form I and Form II. Form II is most preferred.

Amorphous lercanidipine hydrochloride may be prepared by suspending and prefereably dissolving crystalline lercanidipine hydrochloride in an organic solvent at a first temperature in the range from about 30° C. to about 50° C. to form a first solution, adding the first solution to water at a temperature in the range from about 1° C. to about 20° C. to form a precipitate, maintaining the precipitate at a temperature in the range from about 1° C. to about 20° C. , for a period from about 4 to about 24 hours, and recovering the amorphous lercanidipine hydrochloride.

In another embodiment, lercanidipine is provided as a free base. The lercanidipine free base may be present in amorphous form, or as a mixture of amorphous and crystalline forms, wherein the crystalline forms can be of the same polymorph or a combination of two or more polymorphs. Amorphous lercanidipine free base may be prepared by alkalization of a lercanidipine salt in the presence of an organic solvent. The lercanidipine salt may be any salt known in the art, including, but not limited to, those disclosed in U.S. patent application Ser. No. 11/211,769 and/or international application PCT/EP05/009043, which are incorporated herein by reference, or from commercial sources. One particularly preferred lercanidipine salt is lercanidipine hydrochloride.

Alkalization of a lercanidipine salt to yield the free base may be carried out by combining a lercanidipine salt dissolved in an organic solvent with an aqueous medium having a pH in the range from about 9 to about 14. The alkalization reaction may be carried out at temperature from about 0° C. to about 25° C., preferably at a temperature from about 5° C. to about 20° C. Preferably the reaction components are stirred upon combination for a period from about 30 to about 120 minutes, then allowed to stand for a period from about 1 to about 12 hours. Following alkalization the amorphous lercanidipine free base may be separated using any technique known in the art.

Preferably, lercanidipine is present in an amount sufficient to render a therapeutic effect when the modified release composition of the present invention is administered to a patient. Lercanidipine may be present in any amount from about 0.001 to about 0.2 mg per mg of the total composition, and more preferably from about 0.002 mg to about 0.1 mg per mg of the total composition and most preferably 0.005 mg about 0.1 mg per mg of the total composition.

In one embodiment, the lercanidipine is subjected to micronization prior to incorporation into the modified release composition. Lercanidipine crystalline forms can undergo micronization, using any method known in the art. The average size of particles produced by this method are preferably D(50%)2-8 μm, D(90%)<15 μm.

The pharmaceutical composition may optionally include additives, such as for example, pharmaceutically acceptable carriers or diluents, flavorants, sweeteners, preservatives, antioxidants, wetting agents, buffering agents, release controlling agents, dyes, binders, suspending agents, dispersing agents, colorants, excipients, film forming agents, lubricants, plasticizers, edible oils or any combination of two or more of the foregoing. The composition may be related to solid pharmaceutical forms as hard capsule and soft capsules, tablets, coated tablets, or sachets. Suitable pharmaceutically acceptable carriers or diluents include, but are not limited to, ethanol; water; glycerol; propylene glycol; glycerin; diethylene glycol monoethylether, vitamin A and E oils; mineral oil; PPG2 myristyl propionate; magnesium carbonate; potassium phosphate; silicon dioxide; vegetable oil; animal oil; and solketal.

Suitable binders include, but are not limited to, starch; gelatin; corn sweeteners; natural and synthetic gums, such as acacia, tragacanth, vegetable gum, and sodium alginate; carboxymethylcellulose; hydroxypropylmethylcellulose; polyethylene glycol; povidone; waxes; and the like.

Suitable antioxidants include, but are not limited to ascorbic acid, ascobyl palmitate, butylated hydroxyanisole, butylated hydroxytoluene (BHT), monothioglycerol, potassium metabisulfite, propylgallate, tocoferol excipients.

Suitable wetting agents include, but are not limited to polysorbate, sodium lauryl sulfate, sorbitan monolaurate, sorbitan monooleate, sorbitan monopalmitate, sorbitan monostearate.

Suitable additional release modifying agents include, but are not limited to hydroxypropylmethylcellulose, hydroxypropylcellulose, ethylcellulose, hydroxyethylcellulose.

Suitable lubricants include, but are not limited to, sodium oleate, sodium stearate, sodium stearyl fumarate, magnesium stearate, sodium benzoate, sodium acetate, sodium chloride and the like.

Suitable suspending agents include, but are not limited to, bentonite, ethoxylated isostearyl alcohols, polyoxyethylene sorbitol and sorbitan esters, microcrystalline cellulose, aluminum metahydroxide, agar-agar and tragacanth, or mixtures of two or more of these substances, and the like.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable film forming agents include, but are not limited to, hydroxypropylmethylcellulose, ethylcellulose and polymethacrylates.

Suitable plasticizers include, but are not limited to, polyethylene glycols of different molecular weights (e.g., 200-8000 Da), and propylene glycol and triethyl citrate.

Suitable colorants include, but are not limited to, ferric oxide(s), titanium dioxide and natural and synthetic lakes.

Suitable edible oils include, but are not limited to, cottonseed oil, sesame oil, coconut oil and peanut oil.

Examples of additional additives include, but are not limited to, sorbitol, talc, and stearic acid.

In a preferred embodiment, the pharmaceutical composition of the present invention is formed as a solid oral dosage from comprising a polyglycolized glyceride, lercanidipine and a capsule, or other delivery devices suitable for oral administration. Preferably the unit dosage forms comprise a sufficient amount of lercanidipine to impart a therapeutic effect when the dosage form is administered to a patient. More preferably the unit dosage form comprises from about 1 to about 100 mg of lercanidipine, and most preferably about 2 to about 40 mg of lercanidipine.

In another preferred embodiment the pharmaceutical composition of the present invention is formed as a solid dosage form comprising a polyglycolized glyceride, lercanidipine and other suitable excipients in tablets suitable for oral administration. Preferably the unit dosage forms comprise a sufficient amount of lercanidipine to impart a therapeutic effect when the dosage form is administered to a patient. More preferably the unit dosage form comprises from about 1 to about 100 mg of lercanidipine, and most preferably about 2 to about 40 mg of lercanidipine.

In another preferred embodiment the present invention provides a solid oral dosage form comprising a gelatin or hydroxypropylmethylcellulose capsule filled with lercanidipine dissolved or suspended in a Gelucire® material as described herein, preferably Gelucire® 50/13 or Gelucire® 44/14 or a combination thereof. Preferably the ratio of Gelucire® to lercanidipine is from about 1:500 to about 1:5, more preferably from about 1:250 to about 1:10, still more preferably from about 1:200 to about 1:20. Where the solid oral dosage form comprises more than one Gelucire® material, the weight ratio of 50/13:44/14 of within the range of from about 1:99 to about 99:1. In forming the modified released lercanidipine pharmaceutical composition of the invention, the lercanidipine is suspended and preferably dissolved in a melt of polyglycolized glyceride(s). The mixture in the form of a melt comprising polyglycolized glyceride(s) and lercanidipine and/or other excipients dispersed therein may be filled into hard or soft gelatin or hydroxypropylmethylcellulose capsules.

In an additional embodiment, the pharmaceutical composition comprising polyglycolized glyceride and lercanidipine, may be powdered by milling at a low temperature and then incorporated into tablets, beads or beadlets employing conventional procedures. The beads or beadlets may also be formed by the process of prilling where the melt is added dropwise to a non-miscible liquid maintained at a lower temperature.

In another embodiment, the process of the invention involves melting the Gelucire® and heating the molten Gelucire® at a temperature from about 5° C. to about 50° C. above its melting point while stirring. For Gelucire® 50/13, heating is preferably at a temperature from about 55° C. to about 90° C., and more preferably from about 60° C. to about 85° C. For Gelucire® 44/14, heating is preferably at a temperature from about 50° C. to about 80° C., and more preferably from about 55° C. to about 75° C. After heating, the lercanidipine is combined in the molten Gelucire® to make a first mixture. The temperature is maintained during and following mixing, and stirring of the first mixture is continued for a sufficient amount of time to ensure that the admixture is homogeneous, preferably with the lercanidipine dissolved, as judged by visual inspection.

In another embodiment, the process of the invention involves melting the Gelucire® and/or Compritol® and/or Precirol® at a temperature from about 5° C. to about 50° C. above its melting point while stirring. After heating, the lercanidipine is combined with the molten mass to make a first mixture. The temperature is maintained during and following mixing, and stirring of the first mixture is continued for a sufficient amount of time to ensure that the mixture is homogeneous, preferably with the lercanidipine dissolved, as judged by visual inspection.

In another embodiment, the process of the invention involves melting the Gelucire® and/or Compritol® and/or Precirol® at a temperature from about 5° C. to about 50° C. above its melting point while stirring. After heating, the lercanidipine is combined with the molten mass to make a first mixture. The temperature is maintained during and following mixing, and stirring of the first admixture is continued for a sufficient amount of time to ensure that the admixture is homogeneous, preferably with the lercanidipine dissolved, as judged by visual inspection. A suitable polymer, for example Methocel® K 4M, may be added to the mass and stirred until the mixture is homogeneous. The melt is filled into a capsule formed from a suitable polymer, e.g., hydroxypropylmethylcellulose.

### EXAMPLES

The following examples of modified release pharmaceutical compositions and methods of making the same are now disclosed. The examples are illustrative in nature of the various aspects of the invention and are not intended to be limiting in any manner.

### Example 1

### Administration of Modified Release Lercanidipine Capsules to Dogs

The following is a comparative example, comparing the in vivo bioavailability of two different modified release solid unit dosage forms of the present invention with a commercially available immediate release lercanidipine tablets. Commercially available immediate release lercanidipine tablets were obtained from Recordati S.p.A. (Milan, Italy) and comprised 20 mg of lercanidipine per tablet.

Two different modified release solid unit dosage forms were prepared as described below. The composition of the two modified release dosage forms is shown in Table 1. A mixture of lercanidipine free base and Gelucire® was prepared by first melting the Gelucire® by heating to about 70° C. Lercanidipine was added to the heated Gelucire® with continuous mixing until all the added lercanidipine dissolved. The lercanidipine/Gelucire® mixture was then filled into size #0 hard gelatin capsules. Approximately 500 mg of the lercanidipine/Gelucire® was added to each capsule, comprising a total dosage of about 20 mg of lercanidipine. The lercanidipine/Gelucire® filled capsules were than allowed to stand at room temperature to solidify.

TABLE 1
Composition of Modified Release Dosage Forms

Formulation

| | Y1 | Y2 |
|---|---|---|
| Gelucire ® 44/14 | — | 480 mg |
| Gelucire ® 50/13 | 480 mg | — |
| Lercanidipine base | 20 mg | 20 mg |
| Hard Gelatin Capsule size #0 | 1 | 1 |

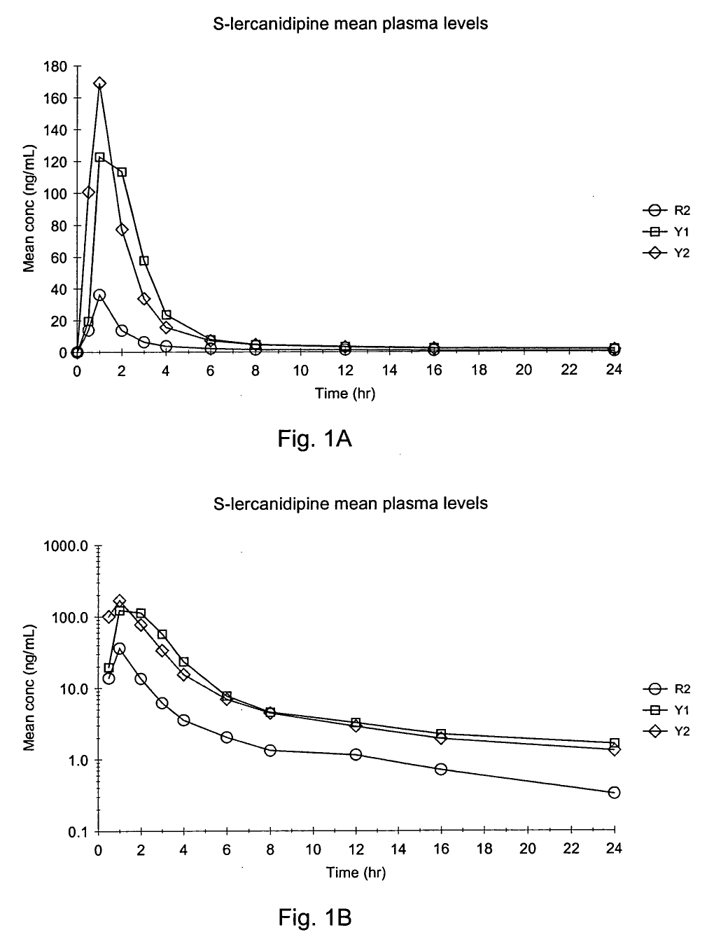

The formulation were tested in male beagle dogs weighing from 8 to 10 Kg. A dose of 40 mg (two capsules) was administered to dogs using a cross-over experimental design. Blood was collected at 0.5 h, 1 h, 2 h, 3 h, 4 h, 6 h, 8 h, 12 h, 16 h, and 24 h after treatment. Results are shown in FIG. 1 and Table 2.

TABLE 2
In vivo pharmacokinetic data

| | | | Cmax | AUClast |
|---|---|---|---|---|
| Treatment | Animal | Tmax (h) | (ng/ml) | (hr * ng/ml) |
| Reference | N | 3 | 3 | 3 |
| | Mean | 1.00 | 36.20 | 74.02 |
| | SD | 0.00 | 17.55 | 34.70 |
| | Min | 1.00 | 16.00 | 34.34 |
| | Median | 1.00 | 44.90 | 89.10 |
| | Max | 1.00 | 47.70 | 98.64 |
| | CV % | 0.00 | 48.50 | 46.90 |
| | Geometric Mean | 1.00 | 32.48 | 67.08 |
| Formulation | N | 3 | 3 | 3 |
| Y1 | Mean | 1.67 | 132.33 | 359.63 |
| | SD | 0.58 | 37.81 | 36.48 |
| | Min | 1.00 | 103.00 | 317.78 |
| | Median | 2.00 | 119.00 | 376.40 |
| | Max | 2.00 | 175.00 | 384.71 |
| | CV % | 34.60 | 28.60 | 10.10 |
| | Geometric Mean | 1.59 | 128.97 | 358.34 |
| Formulation | N | 3 | 3 | 3 |
| Y2 | Mean | 1.00 | 169.00 | 354.64 |
| | SD | 0.00 | 16.00 | 45.90 |
| | Min | 1.00 | 153.00 | 314.16 |
| | Median | 1.00 | 169.00 | 345.26 |
| | Max | 1.00 | 185.00 | 404.51 |
| | CV % | 0.00 | 9.50 | 12.90 |
| | Geometric Mean | 1.00 | 168.49 | 352.70 |

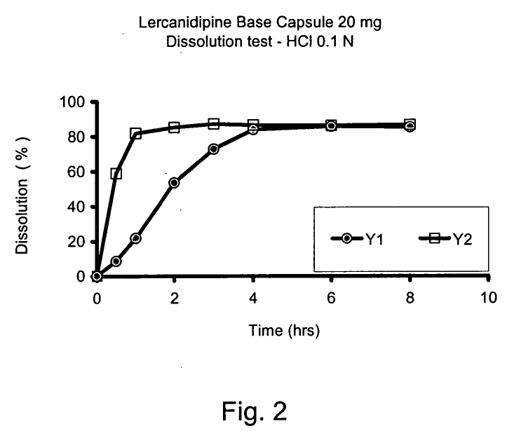

Compared to the commercially available immediate release tablets (reference), the modified release formulations Y1 and Y2 provided for a greater mean plasma concentration of lercanidipine over an extended duration (see FIG. 1). The modified release compositions of the maintained mean plasma concentrations of lercanidipine of greater than 1 ng/ml for duration of the dosing interval, i.e., 24 hours, while the commercially available tablet resulted in a mean plasma concentration of lercanidipine which was less than 0.5 ng/ml after 24 hours. The in vitro dissolution profiles of modified release formulations Y1 and Y2 are shown in FIG. 2.

### Example 2

### Modified Release Lercanidipine Capsules

Different modified release solid unit dosage forms were prepared as described below. The compositions of the modified release dosage forms are shown in Table 3. A mixture of lercanidipine free base, Gelucire®, and Compritol® was prepared by first melting the Gelucire® and Compritol® by heating to about 90° C. Lercanidipine and BHT were added to the heated mass with continuous mixing until all the added lercanidipine dissolved. Methocel® K4M was dispersed into the melted mass under stirring. The lercanidipine/Gelucire®/Compritol®/Methocel® mixture was then filled into size #0 hard gelatin capsules. Approximately 500 mg of the lercanidipine/Gelucire®/Compritol®/Methocel® was added to each capsule, comprising a total dosage of about 20 mg of lercanidipine. The lercanidipine/Gelucire®/Compritol®/Methocel® filled capsules were than allowed to stand at room temperature to solidify.

TABLE 3
Composition of Modified Release Dosage Forms

| | Formulation (in mg/cps) | | | |
|---|---|---|---|---|
| | Y3 | Y4 | Y5 | Y6 |
| Lercanidipine base | 20 | 20 | 20 | 20 |
| Gelucire ® 50/13 | 429.95 | 454.95 | 429.95 | 379.95 |
| Compritol ® 888 | — | 25 | 50 | 50 |
| Methocel ® K 4 M | 50 | — | — | 50 |
| BHT | 0.05 | 0.05 | 0.05 | 0.05 |
| Hard Gelatin Capsule Size #0 | 1 | 1 | 1 | 1 |

### Example 3

### Further Modified Release Lercanidipine Capsules

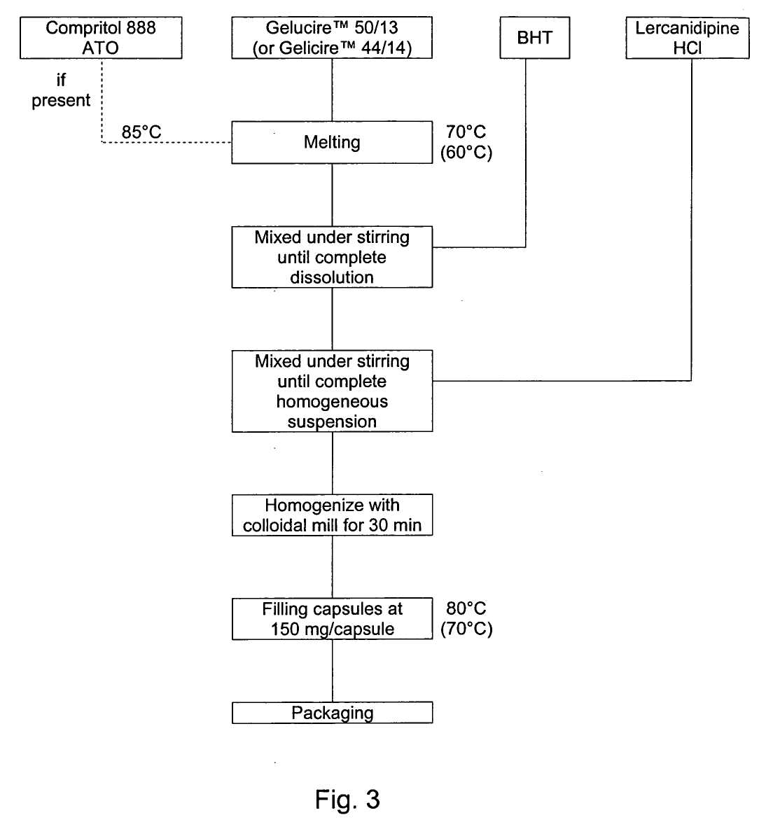

Operating according to the methodology set out in the flowchart shown in FIG. 3 of the accompanying drawings, further formulations according to the invention were prepared. the composition of the formulations is set out below in Table 4.

TABLE 4
Composition of Modified Release Unit Dosage Forms

| | Formulation (in mg/capsule) | | |
|---|---|---|---|
| | Y7 | Y8 | Y9 |
| Lercanidipine HCl | 10.00 mg | 10.00 mg | 10.00 mg |
| Gelucire ® 50/13 | 139.985 mg | — | 125.985 mg |
| Compritol ® 888 | — | — | 14.00 mg |
| ATO | | | |
| Gelucire ® 44/14 | — | 139.985 mg | — |
| BHT | 0.015 mg | 0.015 mg | 0.015 mg |
| TOTAL | 150.00 mg | 150.00 mg | 150.00 mg |

### Example 4

### Administration of Lercanidipine Modified Release Dosage Forms to patients

Modified lercanidipine dosage forms are prepared as described in Table 1, Table 3, or Table 4 with the exception that the dosage forms include 5, 10, or 20 mg lercanidipine. The dosage forms comprising 5, 10, or 20 mg lercanidipine are administered to patients with mild or moderate hypertension once per day at the same time each day for 28 days. Plasma concentration of lercanidipine is measured 24 h after administration of each dose, prior to administration of any subsequent dose. Blood pressure is monitored daily. It is predicted that the plasma levels of lercanidipine measured 24 hours after administration of each dose and immediately prior to administration of a subsequent will be at least 0.5 ng/ml and also predicted that at the end of 28 days blood pressure will be lowered by at least about 15 mm Hg for systolic pressure and/or by about 10 mm Hg for diastolic pressure.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description. Patents, patent applications, publications, product descriptions, and protocols are cited throughout this application, the disclosures of which are incorporated herein by reference in their entireties for all purposes.

1. A modified release lercanidipine pharmaceutical composition comprising at least one waxy substance comprising one or more polyalcohol fatty acyl ester and a therapeutically effective amount of lercanidipine, in a proportion such that after about 24 h following oral administration of the composition to a patient in need of blood pressure reduction, said patient has a mean lercanidipine plasma concentration of greater than or equal to 0.5 ng/ml.

2. The modified release composition according to claim 1 wherein said one or more fatty acyl ester comprises one or more polyalcohol fatty acyl ester selected from the group consisting of polyethylene glycol esters, polypropylene glycol esters, and fatty acid glycerides.

3. The modified release composition according to claim 2 wherein said one or more fatty acyl ester comprises a polyglycolized glyceride.

4. The modified release composition of claim 3 wherein the polyglycolized glyceride is selected from the group consisting of Gelucire® 37/02, 37/06, 42/12, 44/14, 46/07, 48/09, 50/02, 50/13, 33/01, 39/01, 43/01 and 53/10, or a combination thereof.

5. The modified release composition of claim 1 wherein the lercanidipine is lercanidipine free base.

6. A lercanidipine solid oral dosage form comprising the pharmaceutical composition of claim 5 wherein the ratio of the polyglycolized glycerides to lercanidipine is from about 1:500 to about 1:5 (w/w).

7. A lercanidipine solid oral dosage form comprising the pharmaceutical composition of claim 5, wherein said oral dosage form is suitable for once daily oral administration.

8. The lercanidipine solid oral dosage form of claim 7 wherein the total dosage of lercanidipine is from about 1 to about 100 mg per dose.

9. A method of preparing a modified release lercanidipine solid unit dosage form, comprising the steps of:
(i) forming a mixture of one or more polyalcohol fatty acid ester and lercanidipine at a temperature of from about 40° C. to about 90 ° C., and
(ii) filling a capsule with the mixture.

10. The method of claim 9 wherein the polyalcohol fatty acid ester is a mixture of mono-, di-, and triglycerides and polyethylene mono- and diesters.

11. A method of treating hypertension in a patient in need thereof comprising administering to said patient the composition of claim 1, to treat said hypertension.

12. A method of treating hypertension in a patient in need thereof comprising administering to said patient the composition of claim 5, to treat said hypertension.
